(19) United States
(12) Patent Application Publication
(10) Pub. No.: US 2008/0188807 A1
(43) Pub. Date: Aug. 7, 2008

(54) Syringe Assembly Having Re-Use Prevention Features

(76) Inventors: Richard Caizza, Vernon, NJ (US); Robert B. Odell, Franklin Lakes, NJ (US)

Correspondence Address: David W. Highet, VP & Chief IP Counsel;Becton, Dickinson and Company, (Diehl Servilla, LLC), 1 Becton Drive, MC 110, Franklin Lakes, NJ, 07417-1880, US

(21) Appl. No.: 12/026,173

(22) Filed: Feb. 5, 2008

Related U.S. Application Data
(60) Provisional application No. 60/888,166, filed on Feb. 5, 2007.

(51) Int. Cl. A61M 5/50

(52) U.S. Cl. 604/110

(57) ABSTRACT

Medical devices and syringes having reuse prevention features and methods of use thereof are provided. According to one or more embodiments, the syringe includes a barrel and a plunger rod including a stopper and an end wall with at least one portion that is removably engaged with the plunger rod, which prevents the creation of a vacuum between the stopper and barrel. One or more embodiments pertain to a single-use syringe which has a means for separating the plunger rod to prevent disassembly of the syringe prior to use. Methods of using a syringe according to embodiments of the present invention include an aspirating and expelling cycle followed by applying a force to the plunger rod in the distal direction to disable the stopper and prevent the creation of a vacuum between the stopper and syringe barrel.

### CROSS-REFERENCE TO RELATED APPLICATIONS

This application claims the benefit of priority of U.S. Provisional Application No. 60/888,166, filed Feb. 5, 2007, the disclosures of which are hereby incorporated in their entirety by reference thereto.

### FIELD OF THE INVENTION

The present invention relates to syringe assemblies, particularly to syringe assemblies having reuse prevention features, and methods of use thereof.

### BACKGROUND OF THE INVENTION

Throughout the world the multiple-use of hypodermic syringe products which are intended for single-use only, is instrumental in drug abuse and the transfer of contagious diseases. Intravenous drug users who routinely share and reuse syringes are a high risk group with respect to the AIDS virus. Also, the effects of multiple-use are a major concern in some countries where repeated use of syringe products during mass immunization programs may be responsible for the spread many diseases. Reuse of single-use hypodermic syringe assemblies is also instrumental in the spread of drug even in the absence of infection or disease.

Many attempts have been made to remedy this problem. Most notable are early contributions which relied on a specific act to destroy the syringe after use either by using a destructive device or providing a syringe assembly with frangible zones so that syringe can be rendered inoperable by the application of force. Other attempts involve inclusion of additional structure, such as a metal clip in the syringe barrel between the plunger and the barrel to disable the syringe after a predetermined number of strokes. Although costly to manufacture, these syringes work very well to prevent reuse. These syringes are use-specific. A two-stroke syringe which allows one stroke to draw in the fluid and another to deliver the injection cannot be used for a procedure involving reconstituting which requires four strokes. Likewise, a syringe capable of four strokes before locking would still be functional if the drug delivery regimen only involved two strokes.

Further, mass immunization programs usually take place in developing countries where resources are limited and, therefore, the number of people immunized can be increased if the cost of the syringe is reduced.

Accordingly, there is still a need for a cost effective syringe assembly having reuse prevention features which is easy to manufacture and easy to use, like a standard hypodermic syringe.

### SUMMARY OF THE INVENTION

The medical device or syringe assembly of the present invention passively prevents its reuse after the plunger has been bottomed-out in the barrel after the injection cycle. Accordingly, the syringe assembly can be used in a manner similar to ordinary syringe assemblies for filling and injection. One aspect of the present invention pertains to a single-use syringe that is rendered inoperable after the healthcare worker completes the injection of the fluid and bottoms the plunger at the end of the syringe barrel. As the distal end of the plunger approaches and contacts the end wall of the barrel, the disabling feature of the plunger rod is pushed proximally towards the thumbpress into a receiving receptacle or nest that allows venting through the stopper preventing a vacuum from being created between the stopper and the syringe barrel, and therefore preventing the reuse of the syringe. Another aspect of the invention provides a mechanism to disable the syringe if attempts are made to disassemble the plunger rod from the barrel.

An operable syringe assembly having reuse prevention features of the present invention comprises a barrel including a cylindrical side wall having an inside or interior surface defining a chamber for retaining fluid. The barrel includes an open proximal end and a distal end having a distal wall with a passageway therethrough in fluid communication with the chamber. A plunger rod includes a proximal end, a distal end and an elongate main body portion extending between the proximal end and the distal end. The proximal end of the plunger rod includes a thumb press and the distal end of the plunger rod includes a stopper and an end wall. In one or more embodiments, the stopper is in the form of a peripheral edge of the end wall which forms a seal with the interior surface of the barrel and is integrally formed with the plunger rod and/or end wall.

The stopper is slidably positioned in fluid-tight engagement with the inside surface of the barrel for drawing fluid into and driving fluid out of the chamber by movement of the stopper relative to the barrel. In one or more embodiments, the stopper is adapted to form a vacuum with the barrel within the chamber when a force is applied to the plunger rod in the proximal direction. According to one embodiment, the end wall of the plunger rod includes a receptacle which is in fluid communication with the chamber of the barrel through an aperture. In another embodiment, the receptacle has one or more openings which allow fluid communication between the receptacle and the exterior of the medical device.

One or more embodiments provide for a syringe having a means for breaking the vacuum created by the stopper and the barrel. In a specific embodiment, the means for breaking the vacuum is provided by a plunger rod having at least a portion of the end wall that is removably engaged with the distal end of the plunger rod. According to one embodiment, the removal of this portion of the end wall from the distal end of the plunger rod prevents a vacuum from being created between the stopper and syringe barrel. Although the present invention should not be bound by theory, it is believed that if a vacuum cannot be created between the stopper and the syringe barrel, no fluid or medication can be aspirated into the chamber of the barrel and, therefore, the syringe cannot be reused.

In a specific embodiment of the invention, the at least one portion of the end wall that is removably engaged with the distal end of the plunger rod includes a sealing element. The sealing element of one or more embodiments includes a proximal end, a distal end and a sidewall extending between the proximal end and distal end. In a specific embodiment, the sidewall has a first diameter at its proximal end and a second diameter at its distal end, wherein the first diameter is greater than the second diameter. In at least one embodiment, the sealing element is removably engaged with the aperture of the plunger rod and also seals the aperture so that fluid in the chamber will not enter the receptacle. The sealing element of one or more embodiment projects distally outward from the end wall so that when the stopper is in contact with the distal wall of the syringe barrel, the sealing element disengages from the end wall and is pushed or moves at least partially into the receptacle. The removal of the sealing element creates a vent through the end wall or allows the aperture to be at least partially unobstructed. This opening further permits fluid communication between the receptacle and chamber and, therefore, between the chamber and the exterior of the medical device. This vent prevents a vacuum from being formed in the chamber by the action of the fluid-tight engagement of the stopper and the inside surface of the barrel.

One embodiment of the present invention includes means for preventing a vacuum from being reformed between the stopper and the barrel after use of the syringe. In a specific embodiment, this means is provided by a sealing element having one or more grooves disposed near or adjacent to its distal end. According to this embodiment, the grooves are adapted to prevent the sealing element from engaging with the end wall after it has been dislodged or disengaged from the end wall. It is believed that the presence of the one or more grooves creates a vent between the sealing element and the aperture. To reuse such a syringe, user must align the sealing element with the aperture and to move or push the sealing element into the aperture beyond the grooves to create a seal with the aperture.

According to one embodiment of the present invention, the end wall includes a frangible element that connects the at least one removable portion of the end wall or the sealing element to the stopper. The frangible element of one embodiment is adapted to withstand proximal and distal movement of the plunger rod at least two strokes of full proximal and distal movements within the chamber. As used throughout this application, the term “stroke” shall mean a proximal and distal movement of the plunger rod along the length of the syringe barrel. In another embodiment of the invention, the frangible element of the stopper is constructed to break upon sufficient application of a distally directed force to the plunger rod that allows the end wall to contact the distal wall of the barrel.

In one or more embodiments, the plunger rod also includes an annular flange positioned between the end wall and the main body. The plunger rod may also include a means for separating the distal end of the plunger rod from the proximal end of the plunger rod, such as a frangible zone, when a sufficient proximally directed force is applied to the plunger rod. In a specific embodiment, the annular flange further includes an outwardly directed projection disposed on its outer perimeter. According to one embodiment of the present invention, the side wall of the barrel includes a discontinuity on its inside or interior surface which cooperates with the outwardly directed projection of the plunger rod to prevent disassembly of the syringe prior to use. In such embodiments, the side wall has a greater diameter at the portion including the discontinuity than the outwardly directed projection of the plunger rod that allows the plunger rod to be locked inside the barrel of the syringe. In a further embodiment, application of a continuous proximally directed force to the plunger rod causes the frangible zone of the plunger rod to break.

Another aspect of the present invention pertains to a method of using a syringe including the steps of providing a syringe, as described herein, aspirating a predetermined amount of fluid into the chamber by applying a proximally directed force on the plunger, expelling the fluid by applying a distally directed force on the plunger rod and applying a sufficient force in the distal direction to the plunger rod to disable the stopper to prevent the creation of a vacuum between the stopper and syringe barrel upon application of a proximally directed force to the plunger rod. One embodiment of the present invention further includes aspirating the expelled fluid by applying a proximally directed force on the plunger and expelling the fluid a second time by applying a distally directed force on the plunger rod, prior to preventing the creation of a vacuum between the stopper and syringe barrel.

### BRIEF DESCRIPTION OF THE DRAWINGS

FIG. 1 is perspective view of the syringe assembly having reuse prevention features of one embodiment of the present invention;

FIG. 2 is top plan view of the syringe assembly of FIG. 1;

FIG. 3 is a side elevational view of the syringe assembly of FIG. 1;

FIG. 4 is an end view showing the proximal end of the syringe of FIG. 1;

FIG. 5 is a cross-sectional view of the syringe assembly of FIG. 4 taken along line 5-5;

FIG. 6 is a perspective view of the plunger of the syringe assembly according to an embodiment of the present invention;

FIG. 7 is a side elevational view of the plunger of FIG. 6;

FIG. 8 is an end view showing the proximal end of the plunger of FIG. 7;

FIG. 9 is an end view showing the distal end of the plunger of FIG. 7;

FIG. 10 is a cross-sectional view of the plunger of FIG. 7 taken along line 10-10;

FIG. 11 is a cross-sectional view of the plunger of FIG. 8 taken along 11-11;

FIG. 12 is side elevational view of the sealing element of one embodiment of the present invention;

FIG. 13 is an end view showing the distal end of the sealing element of FIG. 12;

FIG. 14 is a side elevational view illustrating the syringe assembly of one embodiment with the frangible zone on the plunger rod broken due to excessive force;

FIG. 15 is a cross-sectional view of the barrel and portions of the plunger of FIG. 14 taken along line 15-15;

FIG. 16 is a cross-section view of the plunger rod of an alternate embodiment of the present invention; and

FIG. 17 is a cross-sectional view of the syringe according to an alternate embodiment of the invention.

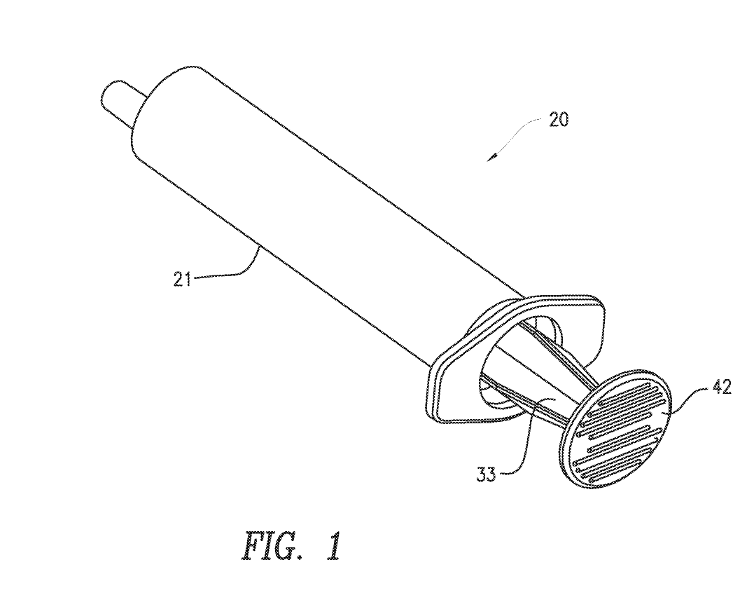

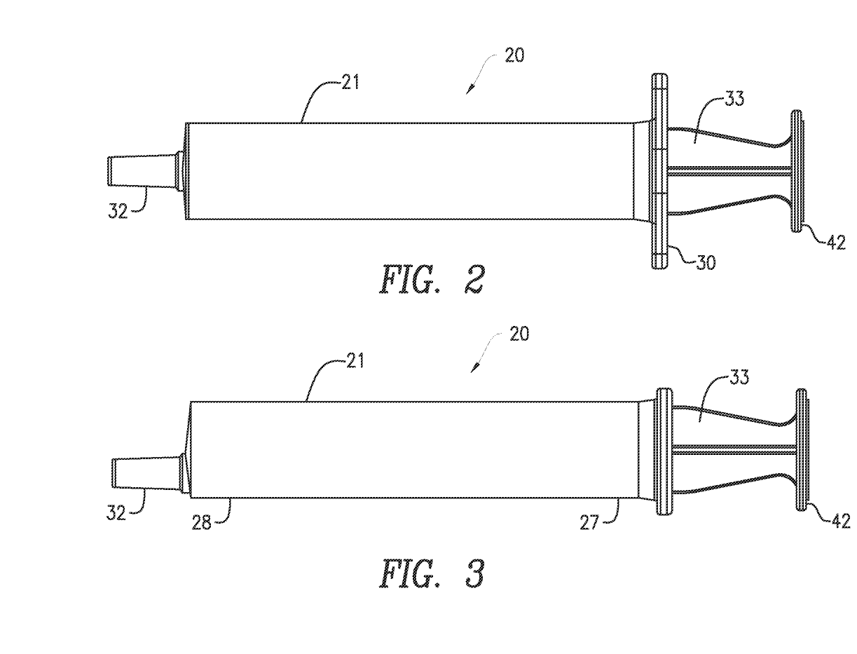

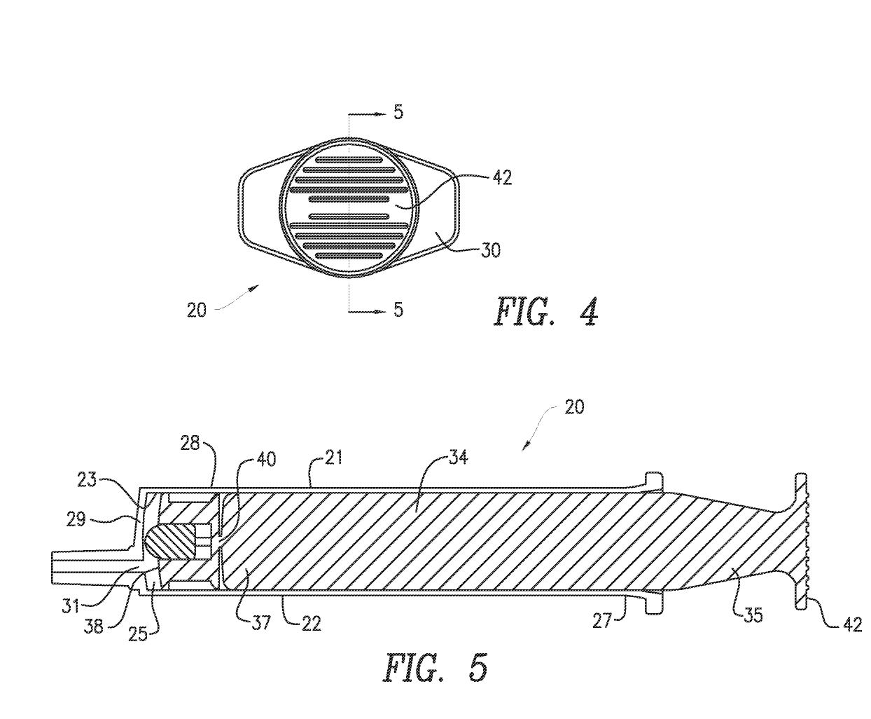

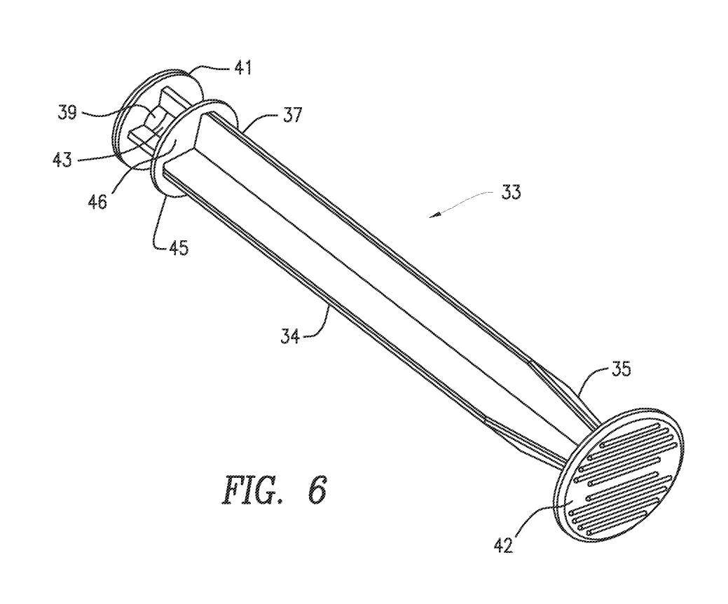

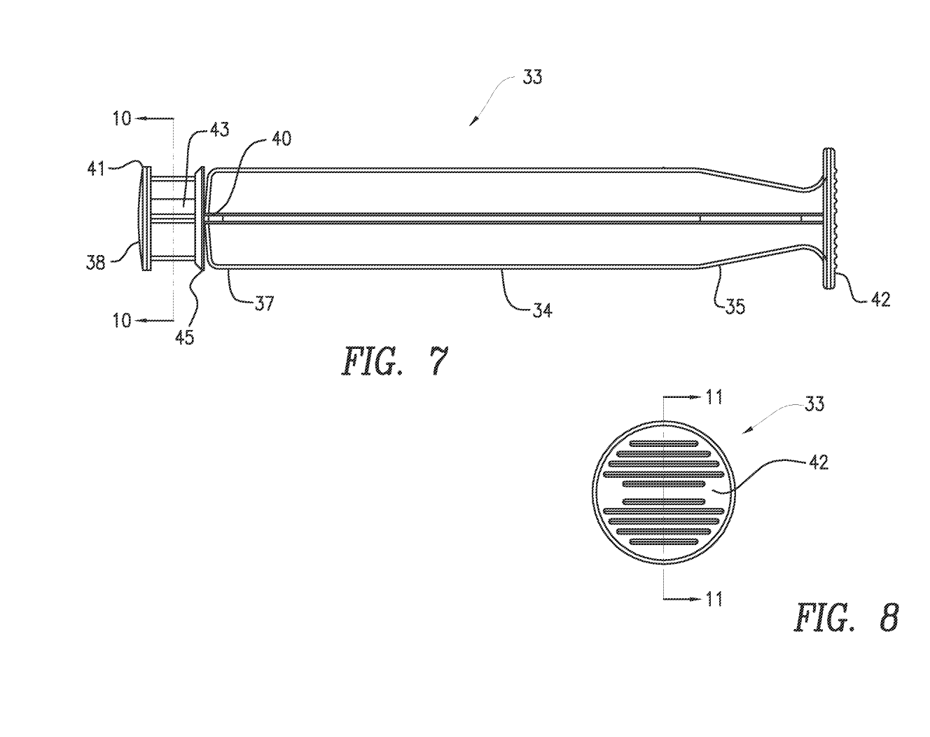

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

For the purpose of the description of the present invention, the term “distal end” is intended to refer to the end furthest from the person holding the syringe assembly, whereas the term “proximal end” is intended to the refer to the end closest to the holder of the syringe assembly.

An operable syringe assembly 20 having reuse prevention features comprises a barrel 21 including a cylindrical sidewall 22 having an inside surface 23 defining a chamber 25 for retaining fluid. The barrel further includes an open proximal end 27 and a distal end 28 including a distal wall 29 having a passageway 31 therethrough in fluid communication with the chamber. The side wall further includes a discontinuity 24 on the inside surface, shown more clearly on FIG. 15.

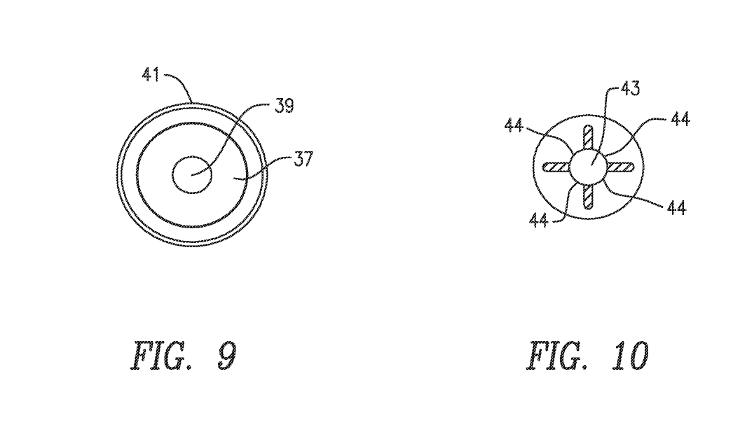

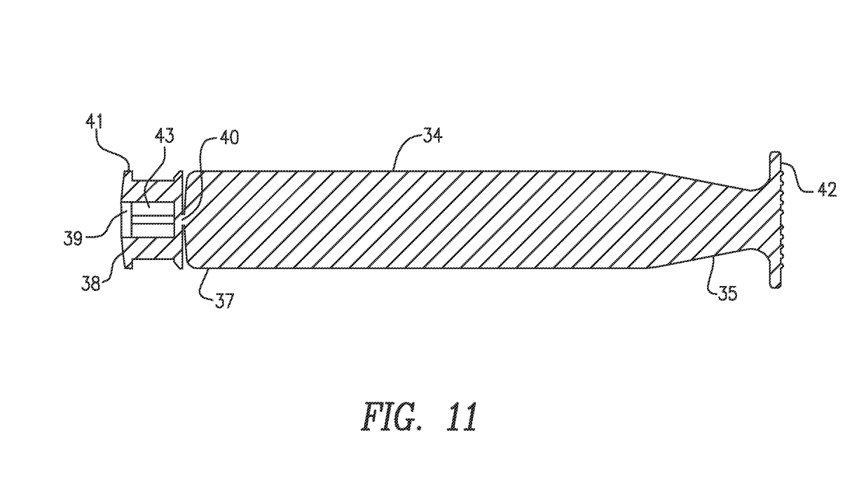

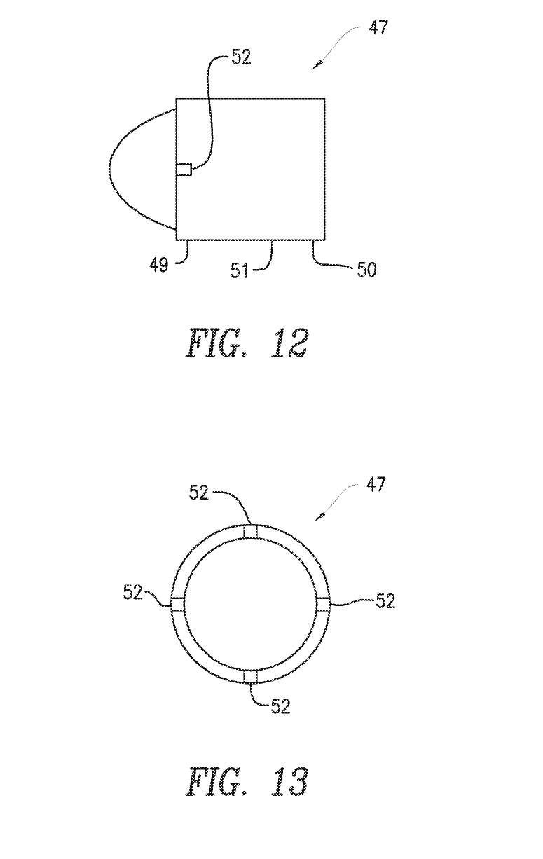

A plunger or plunger rod 33 includes an elongate body portion or main body 34 having a proximal end 35, a distal end 37 having an end wall 38 with an aperture 39 therethrough, and a stopper 41 slidably positioned in fluid-tight engagement with the inside surface of the barrel for drawing fluid into and driving fluid out of the chamber by movement of the stopper relative to the barrel. In the embodiment shown in FIG. 9, the stopper 41 is in the form of a peripheral edge on the end wall 38 which forms a fluid-tight seal with the interior surface of the barrel. According to an alternative embodiment, the stopper can be a separate piece which is removably attached to the distal end of the plunger rod, and includes a peripheral lip that forms a seal with the interior surface of the barrel. The stopper may be comprised of a plastic, elastomeric or other material known in the art suitable for forming a seal.

The elongate body portion of the plunger extends outwardly from the open proximal end of the barrel and includes a thumbpress 42 at its proximal end 35. The plunger further includes a receptacle 43 at its distal end adjacent to end wall 38 and accessible through aperture 39. Receptacle 43 includes at least one opening 44 in its periphery for allowing fluid communication between the receptacle and the exterior of the syringe.

A sealing element 47 having a distal end 49 and a proximal end 50 is removably engaged to end wall 38 and seals aperture 39 so that fluid in the chamber will not enter the receptacle. The distal end of the sealing element projects distally outwardly from the end wall of the plunger so that upon moving the plunger to its distal most position in the barrel, the distal end of the sealing element contacts distal wall 29 of the barrel dislodging sealing element from end wall 38 and moves at least partially into the receptacle. In this position of the receptacle, air may flow back and forth through the aperture, the receptacle and the chamber preventing a vacuum from being formed in the chamber by the action of the fluid-tight engagement of the inside surface of the barrel and the stopper. In one or more embodiments, other means for preventing the formation of a vacuum are provided.

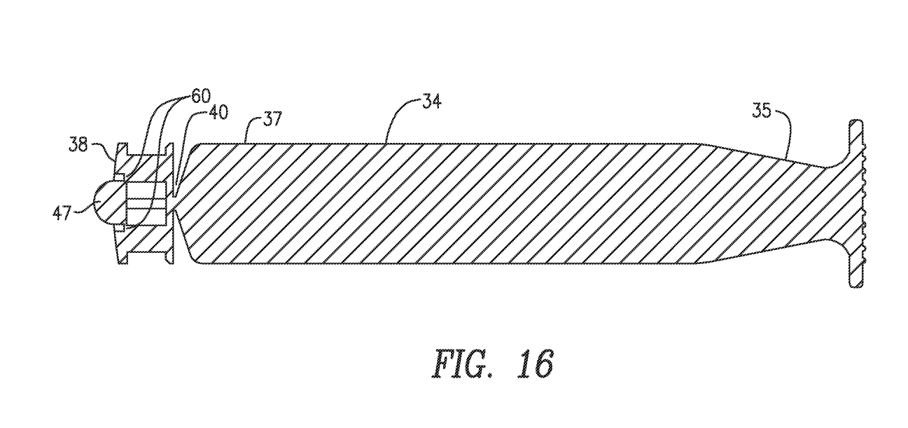

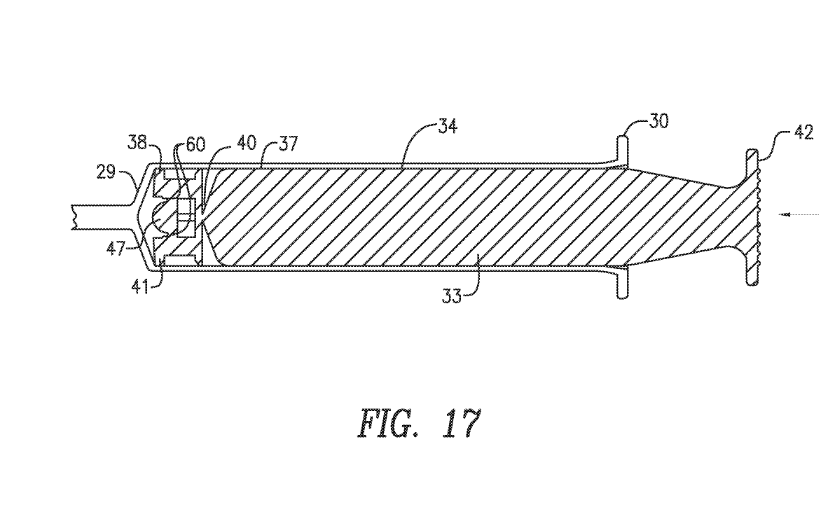

As more clearly shown in FIGS. 16 and 17, an alternate embodiment of the present invention wherein the end wall 39 includes a frangible element 60 that connects the sealing element 47 to the end wall 38. The frangible element 50 is adapted to withstand multiple proximal and distal movements of the plunger rod and is constructed to break upon sufficient application of a distally directed force to the plunger rod 33 that allows the end wall 38 to contact the distal wall of the barrel 29 or approach the distal wall. The breaking of the frangible element will let alert the user of the syringe that the syringe cannot be reused. In one or more embodiments, the end wall does not need to contact the distal wall for the sealing element to disengage or dislodge from the end wall to prevent a vacuum from being formed. As otherwise discussed herein, the sealing element 47 may include one or more grooves 52 on its side wall 51. In such embodiments, the grooves create vents or openings between the sealing element and end wall which prevent formation of a vacuum.

In one embodiment, the end wall and the sealing element are integrally formed to the plunger rod, however, the frangible element allows the plunger rod or syringe to become disabled upon application of sufficient force by the user and prevents a vacuum from being formed between the stopper and the barrel because the stopper cannot be pulled out of the syringe upon application of a proximally directed force on the plunger rod. The frangible element is constructed to allow the syringe to be used with lyophilized medications, while still preventing re-use of the syringe. The material for frangible element selected or the structure and/or shape of the stopper can be modified so that the frangible element can be broken by a user during normal use of the syringe upon application of sufficient force.

According to one embodiment, means for breaking the vacuum formed between the stopper and barrel are also provided.

The plunger rod also includes an outwardly directed projection 45 which in this embodiment is part of annular flange 46. Annular flange 46 is smaller in diameter than the stopper and functions to align the plunger and the barrel. The annular flange does not form a fluid-tight seal with the inside surface of the barrel. The function of outwardly directed projection 45 will be explained in more detail hereinafter. The elongate body portion of the plunger also includes a frangible zone 40 configured to break upon application of excessive force being applied when improperly attempting to reuse the syringe assembly. This feature will also be explained in more detail hereinafter.

Barrel 21 also includes an elongate tip 32 on its distal end. Passageway 31 extends through tip 32 allowing fluid communication between the distal end of the tip and the chamber in the barrel. In this embodiment, tip 32 is frusto-conically shaped to accept a known hypodermic syringe assembly. In the alternative, a hypodermic needle cannula (not shown) can be attached to the tip such as by adhesively bonding the outside of the needle cannula to the interior of the passageway to provide a needle which is permanently attached. In this embodiment, tip 32 is in an eccentric position displaced radially from the longitudinal axis of the barrel. It is also within the purview of the present invention to place the tip centrally along the longitudinal axis of the barrel. Likewise, the aperture on the distal end of the plunger rod containing the sealing element is located along the longitudinal axis of the plunger in this preferred embodiment. However, these elements may be located eccentrically if desired.

It is within the purview of the present invention to include plunger rods and stoppers which are separately formed or integrally formed of the same material or different materials such as in two-material molding, or separately formed of the same or different materials and joined together by mechanical means, adhesives, ultrasonic welding, heat sealing and/or other suitable means. It is understood that the plunger of the present embodiment is merely illustrative of these many possibilities.

In use, the syringe assembly of the present embodiment is provided to the user with its components positioned as best illustrated in FIG. 5. The user may attach a hypodermic needle assembly to the tip on the barrel and fill the syringe from a vial, ampoule or other suitable container using known safe procedures. The liquid is drawn into the chamber of the barrel by holding the barrel and pulling in a proximal direction on thumbpress 42 of the plunger until the desired amount of injectable liquid is contained within the chamber.

An important advantage of the present invention is that the plunger can be moved back and forth along the barrel as many times as necessary to properly fill the syringe barrel. For example, syringe barrel may be filled with sterile water and then sterile water can be injected into a vial containing lyophilized medication which is drawn back into the syringe barrel. Many single-use syringes in the prior art only allow one proximal motion of the plunger with respect to the barrel. Therefore, mixing sterile water and a lyophilized medication as described above is not possible. The liquid in the barrel can now be injected into a patient or delivered in another suitable manner such as through the pierceable septum of a catheter connector. The liquid is injected by holding the barrel, usually in the area of barrel flange 30 and pressing on thumbpress 42 to move the plunger distally in the barrel forcing liquid out through passageway 31 of the barrel. To discharge all of the liquid in the chamber, the user must push the plunger to its distal most position in the barrel wherein end wall 38 of plunger 33 contacts distal wall 29 of the barrel. In the position, distal wall 29 has caused the sealing element to move proximally with respect to the plunger so that it moves further into receptacle 43 where it no longer seals aperture 39. At this point, the syringe assembly can no longer function and is not reusable. Any attempt to draw fluid into the barrel chamber will be unsuccessful since any subatmosphere pressure created by the proximal motion of the stopper in the barrel will be immediately relieved by the venting through the aperture and the opening in the receptacle to the environment. Any attempt to push fluids out of the barrel will also be unsuccessful since the fluid will just pass through the aperture, the opening in the receptacle and into the cavity portion of the barrel proximal of the stopper.

To further ensure that the sealing element no longer seals the aperture in the plunger, grooves 52 be placed in side wall 51 of the sealing element to assure that it will not inadvertently reseal. In the alternative, the structure around the aperture in the plunger rod may be configured with grooves to achieve a similar result.

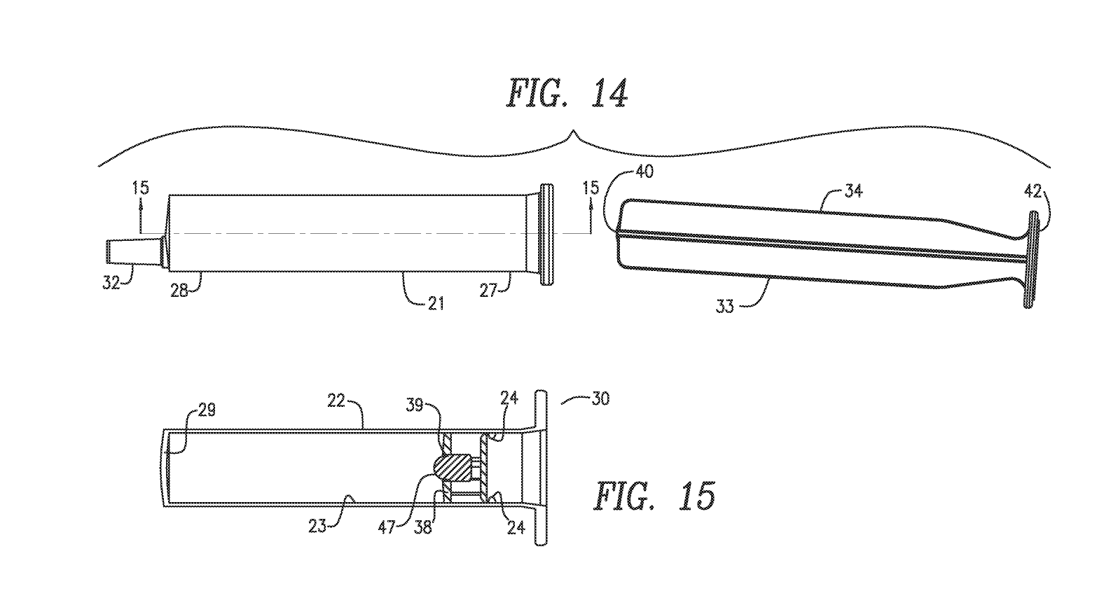

In one embodiment of the present invention, the means for separating or breaking the plunger rod upon application of sufficient force in the proximal direction to the plunger rod. In one embodiment, such means are provided by a frangible zone on the plunger. Any improper attempt to withdraw the plunger, including the stopper, from the barrel by moving the plunger in a proximal direction with respect to the barrel will cause outwardly directed projection 45 on the plunger to engage discontinuity 24 on the inside surface of the cylindrical wall of the barrel as best illustrated in FIG. 15. Further excessive force will result in breaking frangible zone 40 so that part of the plunger falls out of the syringe assembly leaving the stopper in the barrel as best illustrated in FIGS. 14 and 15.

The syringe assembly of the present invention allows each syringe to deliver whatever dose the user requires. The user is not limited to one specific dose. Further the healthcare worker need not perform any additional steps to activate or deactivate the syringe components. The present syringe assembly further eliminates the need for expensive internal mechanisms therefore making syringes more affordable for those who need them most.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

What is claimed is:

1. A medical device comprising:
a barrel including a cylindrical sidewall having an interior surface defining a chamber for retaining fluid, an open proximal end and a distal end including a distal wall having an opening therethrough in fluid communication with said chamber;
an elongate plunger rod including a proximal end, a distal end having a stopper and an end wall, the stopper in fluid-tight engagement with the inside surface of the barrel, and a main body extending between the proximal and distal end, the plunger rod being distally and proximally movable within said chamber, the proximal end including a thumb press; and
at least a portion of the end wall being removably engaged with the distal end of the plunger rod, such that upon removal of said at least portion of the end wall from the distal end of the plunger rod, creation of a vacuum between the stopper and syringe barrel is prevented, thereby preventing reuse of the syringe.

2. The medical device of claim 1, wherein the at least one portion of the end wall is removably engaged with the distal end of the plunger rod by a frangible element, the frangible element adapted to withstand proximal and distal movement of the plunger rod at least two full proximal and distal movements within the chamber and constructed to break upon sufficient application of a distally directed force to the plunger rod that allows the end wall to contact the distal wall of the barrel.

3. The medical device of claim 1, wherein the at least a portion of the end wall includes a sealing element and the stopper comprises a peripheral edge on the end wall which forms a fluid-tight seal with the interior surface of the barrel.

4. The medical device of claim 3, wherein the end wall includes a receptacle and an aperture allowing fluid communication between the receptacle and the chamber, the receptacle including at least one opening for allowing fluid communication between the receptacle and the exterior of the medical device.

5. The medical device of claim 4, wherein the sealing element is removably engaged with and seals the aperture, projecting distally outward from the end wall so that when the stopper is in contact with the distal wall of the syringe barrel, the sealing element disengages from the end wall and moves at least partially into the receptacle, thereby forming a vent through the end wall.

6. The medical device of claim 3, wherein the sealing element has a proximal end, a distal end and a sidewall extending between the proximal end and distal end, the sidewall having a first diameter at its proximal end and a second diameter at its distal end, the first diameter being greater than the second diameter.

7. The medical device of claim 3, wherein the side wall of the sealing element further comprises one or more grooves disposed adjacent to the distal end of the sealing element, the grooves adapted to prevent the sealing element from engaging with the end wall after disengaging from the end wall.

8. The medical device of claim 1, wherein the plunger rod further includes an annular flange disposed between the end wall and the main body, an outwardly directed projection disposed on the outer perimeter of the annular flange, and a frangible zone.

9. The medical device of claim 8, wherein the interior surface of the barrel further comprises a discontinuity, the discontinuity having a diameter greater than the diameter of the outwardly directed projection of the plunger rod to lock the plunger rod in the barrel.

10. The medical device of claim 9, wherein application of a continuous proximally directed force to the plunger rod causes the frangible zone of the plunger rod to break.

11. A single-use medical device comprising:
a barrel including a cylindrical sidewall having an interior surface defining a chamber for retaining fluid, an open proximal end and a distal end including a distal wall having an opening therethrough in fluid communication with said chamber;
an elongate plunger rod including a proximal end, a distal end having an end wall and a stopper in fluid-tight engagement with the inside surface of the barrel, and a main body extending between the proximal and distal end, the proximal end including a thumb press, the stopper adapted to form a vacuum with the barrel within the chamber; and
means for breaking the vacuum, the plunger rod being distally and proximally movable within said chamber for at least two aspiration and expulsion cycles.

12. The single-use syringe of claim 11 further comprising means for separating the distal end of the plunger rod from the proximal end of the plunger rod upon application of sufficient proximally directed force on the plunger rod.

13. The single-use syringe of claim 11, wherein the end wall further comprises a frangible element for connecting the end wall to the means for breaking the, the frangible element is adapted to break upon sufficient distally directed force on the plunger rod.

14. The single-use syringe of claim 11 further comprising means for preventing the vacuum from being reformed.

15. A method of using a syringe comprising:
providing a syringe having a barrel including a cylindrical sidewall having an interior surface defining a chamber for retaining fluid, an open proximal end and a distal end including a distal wall having an opening therethrough in fluid communication with said chamber; and an elongate plunger rod including a proximal end, a distal end having an end wall and a stopper in fluid-tight engagement with the inside surface of the barrel, and a main body extending between the proximal and distal end, the plunger rod being distally and proximally movable within said chamber, the proximal end including a thumb press;
aspirating a predetermined amount of fluid into the chamber by applying a proximally directed force on the plunger;
expelling the fluid by applying a distally directed force on the plunger rod; and
applying a sufficient force in the distal direction to the plunger rod to disable the stopper to prevent the creation of a vacuum between the stopper and syringe barrel upon application of a proximally directed force to the plunger rod.

16. The method of claim 15, wherein at least a portion of the end wall is removably engaged with the distal end of the plunger rod, such that upon removal of said at least portion of the end wall from the distal end of the plunger rod, creation of a vacuum between the stopper and syringe barrel is prevented upon application of a proximally directed force on the plunger rod, thereby preventing reuse of the syringe.

17. The method of claim 15, further comprising aspirating the expelled fluid by applying a proximally directed force on the plunger and expelling the fluid a second time by applying a distally directed force on the plunger rod, prior to preventing the creation of a vacuum between the stopper and syringe barrel.

18. The method of claim 16, wherein the at least a portion of the end wall is a sealing element and the end wall further includes a receptacle and an aperture allowing fluid communication between the receptacle and the chamber, the receptacle including at least one opening for allowing fluid communication between the receptacle and the exterior of the medical device.

19. The method of claim 18, wherein the sealing element projects distally outward from the end wall so that when the stopper is in contact with the distal wall of the syringe barrel, the sealing element disengages from the end wall and moves at least partially into the receptacle, thereby forming a vent through the end wall which prevents the creation of a vacuum.

20. The method of claim 17, wherein the end wall includes a frangible element that, when broken, prevents a vacuum from being formed between the stopper and syringe barrel, the frangible element is adapted to withstand multiple proximal and distal movements of the plunger rod where the end wall does not contact the distal wall of the barrel and to break upon application of a sufficient distally directed force to the plunger rod such that the end wall is in contact with the distal wall of the barrel.
